(19) United States
(12) Patent Application Publication
(10) Pub. No.: US 2026/0085040 A1
(43) Pub. Date: Mar. 26, 2026

(54) AMINO ACID AND CARBOHYDRATE PSILOCIN DERIVATIVES

(71) Applicant: BIONXT SOLUTIONS INC., Vancouver (CA)

(72) Inventors: Raimar LOEBENBERG, Edmonton (CA); Chuanjun GAO, Edmonton (CA); Rong LING, Edmonton (CA)

(21) Appl. No.: 19/110,802

(22) PCT Filed: Sep. 11, 2023
(86) PCT No.: PCT/CA2023/051206

§ 371 Date: Mar. 11, 2025

Related U.S. Application Data
(60) Provisional application No. 63/405,809, filed on Sep. 12, 2022.

(51) Int. Cl. C07D 209/16; C07D 209/20; C07D 403/12; C07H 17/02

(52) U.S. Cl. CPC C07D 209/16; C07D 209/20; C07D 403/12; C07H 17/02

(57) ABSTRACT

This disclosure relates to heterocyclic compounds of Formula (I) as well as the preparation and use thereof. As contemplated herein, heterocyclic compounds of Formula (I) can be any one of a carbohydrate conjugate and an amino acid conjugate. The heterocyclic compounds of Formula (I) may be used for the treatment of neuropsychiatric, and neurodegenerative, neuroinflammatory and pain disorders including depression, as well as tobacco, opiate, and cocaine addiction, alcoholism, post-traumatic stress disorder (PTSD), and pain syndromes including cluster headaches and chemotherapy induced peripheral neuropathy.

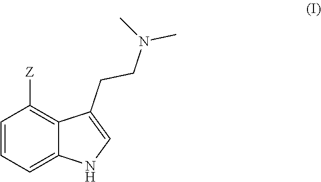

### TECHNICAL FIELD

This present disclosure relates to heterocyclic compounds and methods of preparing the same. This present disclosure also relates to uses of heterocyclic compounds as selective agents at serotonin receptors.

### BACKGROUND

Psilocybin is a naturally occurring psychedelic compound produced by more than 200 species of mushrooms collectively known as “psilocybin mushrooms”. As a prodrug, psilocybin is quickly metabolized by the body to generate the bioactive compound psilocin, which has mind-altering effects not unlike those produced by other psychedelics such as lysergic acid diethylamide (LSD), mescaline, and N,N-dimethyltryptamine (DMT). These effects include, inter alia, euphoria, visual and mental hallucinations, changes in perception, a distorted sense of time, and spiritual experiences, and can also include possible adverse reactions such as nausea and panic attacks.

As agonists of the 5-HT_{2A} and 5-HT_{2C} receptors, psilocybin and psilocin have been recognized for their therapeutic potential. Since 5-HT_{2A} receptor activation appears to increase locomotor activity, whereas 5-HT_{2C} receptor activation appears to decrease locomotor activity, compounds possessing varying degrees of 5-HT_{2A} and 5-HT_{2C} activity will show varying levels of psychedelic activity (Halberstadt A L, van der Heijden I, Ruderman M A, Risbrough V B, Gingrich J A. Geyer M A, Powell S B, Neuropsychopharmacology, 2009, 34(8):1958-67). While psilocybin, along with other psychedelic drugs, were explored more than 60 years ago by Hofmann and co-workers at Sandoz (see for example, Hofmann, A., Troxler, F. U.S. Pat. Nos. 3,075,992; 3,078,214), clinical investigations into these drugs waned substantially by the early 1970s—particularly after these drugs were placed on Schedule 1 of the Controlled Substance Act in the United States of America. Notwithstanding their listing as controlled substances in certain jurisdictions however, research into psilocybin and other psychedelic drugs never fully stopped, and recent clinical investigations have led to a revived interest in the potential application of psychedelic drugs (including psilocybin) in evolving medical areas, such as the treatment of central nervous system (CNS) diseases. CNS diseases include both difficult-to-treat mental health disorders (Daniel J, Haberman M. Clinical potential of psilocybin as a treatment for mental health conditions. Ment. Health Clin. 2017, 7(1), 24-8), such as treatment resistant depression or drug resistant depression, and neurological disorders such as cluster headaches.

As a phosphate phenolic prodrug, psilocybin, once ingested, is rapidly metabolized to the bioactive constituent psilocin; psilocin then acts on serotonin receptors in the brain. The 5-hydroxytryptamine receptors (5-HT) receptors, or serotonin receptors, are a group of G protein-coupled receptors and ligand-gated ion channels found in both the central and peripheral nervous systems. They mediate both excitatory and inhibitory neurotransmission. The 5-HT receptors are activated by the neurotransmitter 5-hydroxytryptamine more commonly known as serotonin, which is the natural ligand.

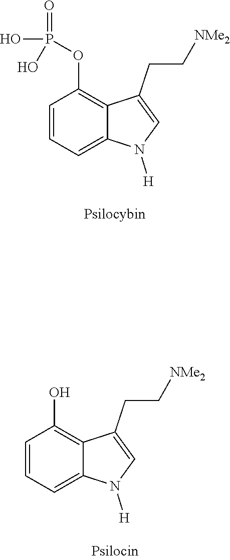 [SMILES: CN(C)CCc1c[nH]c2cccc(OP(=O)(O)O)c12 ; CN(C)CCc1c[nH]c2cccc(O)c12]

Other prodrug linkages are taught in the literature, such as Wiemer et al. Top Curr Chem. 2015; 360: 115-160 and Mahato et al. Adv Drug Deliv Rev. 2011 Jul. 18; 63(8): 659-670.

In a proof-of-concept study for psilocybin for use in treating alcohol dependence involving 10 patients with a diagnosis of alcohol dependence per the DSM-IV, a significant decrease in alcohol use post psilocybin administration among the patients was observed (Bogenschutz M P, et al., Psilocybin-assisted treatment for alcohol dependence: a proof-of-concept study. J. Psychopharmacol. 2015, 29(3), 289-99). In a study on the treatment of tobacco addiction with psilocybin, researchers found that eighty percent of participants showed biologically verified 7-day point-prevalence abstinence at 6-month follow up. The 12-month follow-up showed 67% of the participants were biologically verified as being abstinent. A follow-up 2.5 years after the target quit date showed 75% were abstinent (Johnson, M. W., and Griffiths, R. R. (2017) Potential Therapeutic Effects of Psilocybin. Neurotherapeutics 14, 734).

Notwithstanding the fact that psilocybin has recognized therapeutic potential for treating certain CNS diseases and disorders, its therapeutic potential may be limited owing to low gut, cornea, and skin permeability. As such, there is a need for alternative prodrugs of psilocin.

### SUMMARY

According to a part of the present disclosure, there are chemical entities of Formula I,

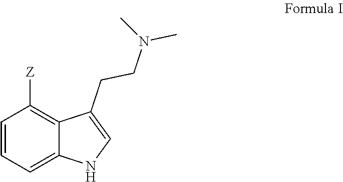

wherein Z is defined hereinafter.

The chemical entities of Formula I may be selected from the group consisting of an amino acid ester and a carbohydrate conjugate. Z may be selected from the group consisting of C₆H₁₁O₆—, C₁₂H₂₁O₁₁—, C₅H₉O₅—, and C₅H₉O₄—. Z may be selected from the group consisting of C₆H₁₃N₄O₂—, C₆H₉N₃O₂—, C₆H₁₃N₂O₂—, C₄H₆NO₄—, C₅H₈NO₄—, C₃H₇NO₃—, C₄H₈NO₃—, C₄H₇N₂O₃—, C₅H₉N₂O₃—, C₃H₆NO₂X—, C₂H₄NO₂—, C₅H₈NO₂—, C₅H₈NO₃—, C₃H₆NO₂—, C₅H₁₀NO₂—, C₆H₁₂NO₂—, C₅H₁₀NO₂S—, C₉H₁₀NO₂—, C₉H₁₀NO₃—, and C₁₁H₁₁N₂O₂—, and derivatives of any one thereof. X may be selected from the group consisting of S and Se.

The chemical entities of Formula I may be selected from the group consisting of glucose conjugates, allose conjugates, altrose conjugates, mannose conjugates, gulose conjugates, idose conjugates, galactose conjugates, talose conjugates, psicose conjugates, fructose conjugates, sorbose conjugates, tagatose conjugates, and derivatives of any one thereof.

The chemical entities of Formula I may be selected from the group consisting of trehalose conjugates, sucrose conjugates, and derivatives of any one thereof.

The chemical entities of Formula I may be selected from the group consisting of ribose conjugates, arabinose conjugates, xylose conjugates, lyxose conjugates, ribulose conjugates, xylulose conjugates, and derivatives of any one thereof.

The chemical entities of Formula I may be selected from the group consisting of ribose conjugates, arabinose conjugates, xylose conjugates, lyxose conjugates, ribulose conjugates, xylulose conjugates, and derivatives of any one thereof.

The chemical entities of Formula I may be selected from the group consisting of a proline ester, a valine ester, and derivatives of any one thereof.

The chemical entity of Formula I may be a valine ester or derivative thereof.

This summary does not necessarily describe the entire scope of all aspects of the disclosure. Other aspects, features and advantages will be apparent to those of ordinary skill in the art upon review of the following description of specific embodiments.

### BRIEF DESCRIPTION OF DRAWINGS

In the accompanying drawings, which illustrate one or more examples of chemical entities of Formula I:

FIG. 1 is a graph that depicts the plasma concentration, as determined by computer modelling, of psilocybin and an alanine conjugate over time.

FIG. 2 is a graph that depicts the plasma concentration, as determined by computer modelling, of psilocybin and a deoxyribose conjugate over time.

FIG. 3 is a graph that depicts the plasma concentration, as determined by computer modelling, of psilocybin and a fructose conjugate over time.

FIG. 4 is a graph that depicts the plasma concentration, as determined by computer modelling, of psilocybin and a glucose conjugate over time.

FIG. 5 is a graph that depicts the plasma concentration, as determined by computer modelling, of psilocybin and a glucoronic acid conjugate over time.

FIG. 6 is a graph that depicts the plasma concentration, as determined by computer modelling, of psilocybin and a glycine conjugate over time.

FIG. 7 is a graph that depicts the plasma concentration, as determined by computer modelling, of psilocybin and a hydroxyproline conjugate over time.

FIG. 8 is a graph that depicts the plasma concentration, as determined by computer modelling, of psilocybin and a leucine conjugate over time.

FIG. 9 is a graph that depicts the plasma concentration, as determined by computer modelling, of psilocybin and a methionine conjugate over time.

FIG. 10 is a graph that depicts the plasma concentration, as determined by computer modelling, of psilocybin and a phenylalanine conjugate over time.

FIG. 11 is a graph that depicts the plasma concentration, as determined by computer modelling, of psilocybin and a proline conjugate over time.

FIG. 12 is a graph that depicts the plasma concentration, as determined by computer modelling, of psilocybin and a ribose conjugate over time.

FIG. 13 is a graph that depicts the plasma concentration, as determined by computer modelling, of psilocybin and a serine conjugate over time.

FIG. 14 is a graph that depicts the plasma concentration, as determined by computer modelling, of psilocybin and a sucrose conjugate over time.

FIG. 15 is a graph that depicts the plasma concentration, as determined by computer modelling, of psilocybin and a trehalose conjugate over time.

FIG. 16 is a graph that depicts the plasma concentration, as determined by computer modelling, of psilocybin and a tryptophane conjugate over time.

FIG. 17 is a graph that depicts the plasma concentration, as determined by computer modelling, of psilocybin and a valine conjugate over time.

### DETAILED DESCRIPTION

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

As used herein and unless otherwise specified, the term “about”, when used to describe a recited value, means within 10% of the recited value.

As used herein and unless otherwise specified, the term “amino acid conjugate” refers to an amino acid or derivative thereof that has been conjugated into an ester, wherein said “amino acid conjugate” has a chemical structure of Formula I. For example, non-limiting examples of “amino acid conjugates” include a “valine conjugates”, and non-limiting examples of “valine conjugates” include the following:

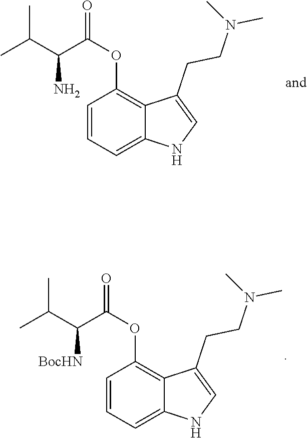

The term “amino acid conjugate” may be used interchangeably with “amino acid ester” in this disclosure.

As used herein and unless otherwise specified, the term “amino acid ester” refers to an amino acid or derivative thereof whose carboxylic acid group has been converted to an ester, wherein said “amino acid ester” has a chemical structure of Formula I. For certainty, non-limiting examples of “amino acid esters” include “valine esters”, and non-limiting examples of “valine esters” include the following:

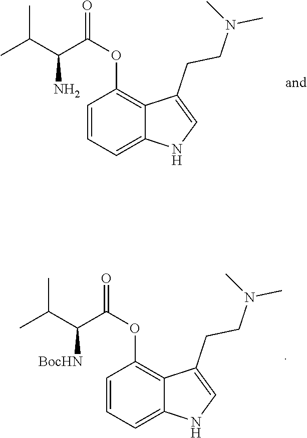

The term “amino acid ester” may be used interchangeably with “amino acid conjugate” in this disclosure.

As used herein and unless otherwise specified, the term “AUC” means “area under the curve” which, in pharmacokinetics, refers to the area under the graphical plot of plasma concentration of a chemical compound versus time after dosage, gives insight into the extent of a biological system's exposure to a chemical compound and a chemical compound's clearance rate from a biological system, and is measured in ng*hours/mL in this disclosure.

As used herein and unless otherwise specified, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form (e.g., a polymorph, pseudohydrate, hydrate, or solvate). Similarly, the term encompasses the compound whether provided in solid form or otherwise. Unless otherwise specified, all statements made herein regarding “compounds” apply to the associated chemical entities, as defined.

As used herein and unless otherwise specified, the term “CL” means “clearance” which is a pharmacokinetic measurement of the volume of plasma from which a substance is completely removed from a biological system per unit time. In this disclosure, “CL” or “clearance” is measured in litres per hour.

As used herein and unless otherwise specified, the terms “comprising”, “having”, “including”, “containing”, and grammatical variations thereof, are inclusive or open-ended and do not exclude additional, un-recited elements and/or method steps. For example, “A includes 1, 2, and 3” means that A includes but is not limited to 1, 2, and 3.

As used herein and unless otherwise specified, the term “consisting essentially of” when used herein in connection with a composition, use, or method, denotes that additional elements, method steps or both additional elements and method steps may be present, but that these additions do not materially affect the manner in which the recited composition, method, or use functions.

As used herein and unless otherwise specified, the term “consisting of” when used herein in connection with a composition, use, or method, excludes the presence of additional elements and/or method steps.

As used herein and unless otherwise specified, the term “carbohydrate conjugate” refers to a carbohydrate, or derivative thereof, that has been conjugated into an ether, wherein said “carbohydrate conjugate” has a chemical structure of Formula I. Non-limiting examples of “carbohydrate conjugates” include glycosides. Non-limiting example of a “carbohydrate conjugate” includes a “glucose conjugate”, as depicted as follows:

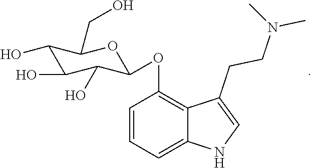 [SMILES: CN(C)CCc1c[nH]c2cccc(OC3OC(CO)C(O)C(O)C3O)c12]

As used herein and unless otherwise specified, the term “Cₘₐₓ” refers to maximum plasma concentration of a chemical compound, as measured in ng/mL in this disclosure.

As used herein and unless otherwise specified, the term “GP” refers to the permeability of a chemical compound through the gut, as measured in cm/s×104 in this disclosure.

As used herein and unless otherwise specified, the term “isotopologue” refers to a species that differs from a specific compound only in the isotopic composition thereof. For example, all hydrogen atoms in a compound are independently of natural isotopic composition or of any isotopic composition enriched or depleted in one or both of the heavy isotopes, ²H (D, deuterium) and ³H (T, tritium), ranging from a depletion to zero % to an enrichment to 100%.

As used herein and unless otherwise specified, the term “MW” refers to molecular weight of a chemical compound, as measured in grams in this disclosure.

As used herein and unless otherwise specified, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio.

Pharmaceutically acceptable salts of the compounds provided in this disclosure include salts derived from suitable inorganic and organic acids and bases.

As used herein and unless otherwise specified, the term “subject” includes a mammal (e.g., a human, and in some embodiments including prenatal human forms). In some embodiments, a subject suffers from a relevant disease, disorder, or condition. In some embodiments, a subject is susceptible to a disease, disorder, or condition. In some embodiments, a subject displays one or more symptoms or characteristics of a disease, disorder, or condition.

In some embodiments, a subject does not display any symptom or characteristic of a disease, disorder, or condition. In some embodiments, a subject is a mammal with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human that is between two and 18 years of age. In some embodiments, an adult refers to a human that is eighteen years of age or older.

As used herein and unless otherwise specified, the phrase “such as” is intended to be open-ended and not limiting.

Reference to specific moieties, functional groups, or substituents contemplates (where applicable) tautomers thereof.

Unless otherwise stated, structures depicted herein include all isomeric (e.g., enantiomeric, diastereomeric, and geometric (or conformational)) forms of the structure (e.g., the R and S configurations for each asymmetric center, Z and E double bond isomers, and Z and E conformational isomers). Unless otherwise stated, the compounds disclosed, taught, or otherwise suggested in this disclosure contemplate all single stereochemical isomers as well as enantiomeric, diastereomeric, and geometric (or conformational) mixtures thereof. Unless otherwise stated, the compounds disclosed, taught, or suggested in this disclosure contemplate all tautomeric forms thereof. Additionally, unless otherwise stated, structures depicted herein include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (²H) may afford certain therapeutic advantages resulting from greater metabolic stability, for example, increase in vivo half-life, or reduced dosage requirements.

Chemical entities described herein are further illustrated by the classes, subclasses, and species disclosed herein. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. In this disclosure, any atom not specifically designated as a particular isotope is meant to represent any stable isotope of that atom.

According to a part of this disclosure, there are chemical entities (including any isotopologue and pharmaceutically acceptable salt thereof) of Formula I:

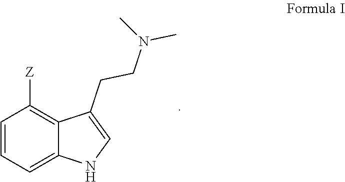

wherein the chemical compound is any one of a carbohydrate conjugate and an amino acid conjugate.

The carbohydrate conjugate can be selected from the group consisting of a glucose conjugate, an allose conjugate, an altrose conjugate, a mannose conjugate, a gulose conjugate, an idose conjugate, a galactose conjugate, a talose conjugate, a psicose conjugate, a fructose conjugate, a sorbose conjugate, a tagatose conjugate, a trehalose conjugate, a sucrose conjugate, a ribose conjugate, an arabinose conjugate, and a deoxyribose conjugate. In some embodiments, the carbohydrate conjugate can be selected from the group consisting of a derivative of any one of the conjugates identified in the foregoing sentence of this paragraph.

The carbohydrate conjugate can be a chemical entity of Formula I wherein Z is selected from the group consisting of C₆H₁₁O₆—, C₁₂H₂₁O₁₁—, C₅H₉O₅—, and C₅H₉O₄—. For example, the carbohydrate conjugate can be:

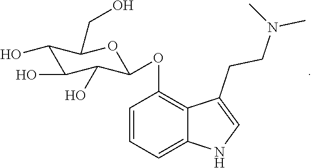 [SMILES: CN(C)CCc1c[nH]c2cccc(OC3OC(CO)C(O)C(O)C3O)c12]

In some embodiments, the carbohydrate conjugate can be selected from the group consisting of a derivative of any one of the conjugates identified in the foregoing sentences of this paragraph.

Non-limiting examples of C₆H₁₁O₆— include glucose, allose, altrose, mannose, gulose, idose, galactose, talose, psicose, fructose, sorbose, and tagatose groups. Non-limiting examples of C₁₂H₂₁O₁₁— include trehalose and sucrose groups. Non-limiting examples of C₅H₉O₅— include ribose, arabinose, xylose, lyxose, ribulose, and xylulose groups. Non-limiting examples of C₅H₉O₄— include deoxyribose groups.

The amino acid conjugate can be selected from the group consisting of an arginine conjugate, a histidine conjugate, a lysine conjugate, an aspartic acid conjugate, a glutamic acid conjugate, a serine conjugate, a threonine conjugate, an asparagine conjugate, a glutamine conjugate, a cysteine conjugate, a glycine conjugate, a proline conjugate, a hydroxyproline conjugate, an alanine conjugate, a valine conjugate, an isoleucine conjugate, a leucine conjugate, a methionine conjugate, a phenylalanine conjugate, a tyrosine conjugate, and a tryptophan conjugate. In some embodiments, the amino acid conjugate can be selected from the group consisting of a derivative of any one of the conjugates identified in the foregoing sentence of this paragraph.

The amino acid conjugate can be selected from the group consisting of the proline conjugate and the valine conjugate. Non-limiting examples of valine conjugates include:

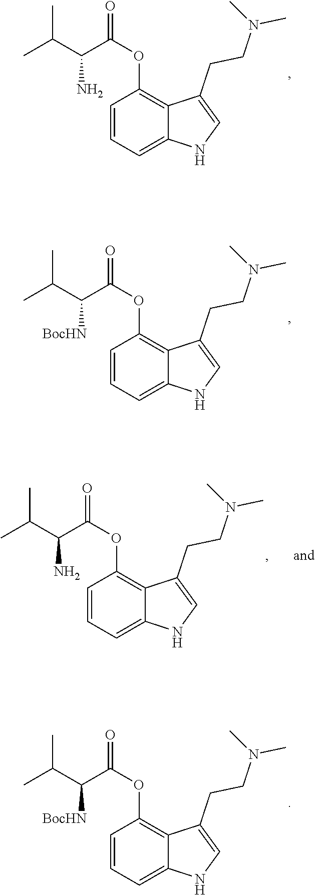

In some embodiments, the amino acid conjugate can be selected from the group consisting of a derivative of any one of the conjugates identified in the foregoing sentences of this paragraph.

The amino acid conjugate can be a chemical compound of Formula I wherein Z is selected from the group consisting of C₆H₁₃N₄O₂—, C₆H₉N₃O₂—, C₆H₁₃N₂O₂—, C₄H₆NO₄—, C₅H₈NO₄—, C₃H₇NO₃—, C₄H₈NO₃—, C₄H₇N₂O₃—, C₅H₉N₂O₃—, C₃H₆NO₂X—, C₂H₄NO₂—, C₅H₈NO₂—, C₅H₈NO₃—, C₃H₆NO₂—, C₅H₁₀NO₂—, C₆H₁₂NO₂—, C₅H₁₀NO₂S—, C₉H₁₀NO₂—, C₉H₁₀NO₃—, and C₁₁H₁₁N₂O₂—, and wherein X can be selected from the group consisting of S and Se. In some embodiments, the amino acid conjugate can be selected from the group consisting of a derivative of any one of the conjugates identified in the foregoing sentence of this paragraph.

Amino acid conjugates include those conjugates comprising a protecting group on the amino functional group, such protecting group being removed to form the amino acid conjugate that would be introduced into the targeted biological system. Protecting groups can include Boc, CBz, and the like.

### Pharmacokinetics

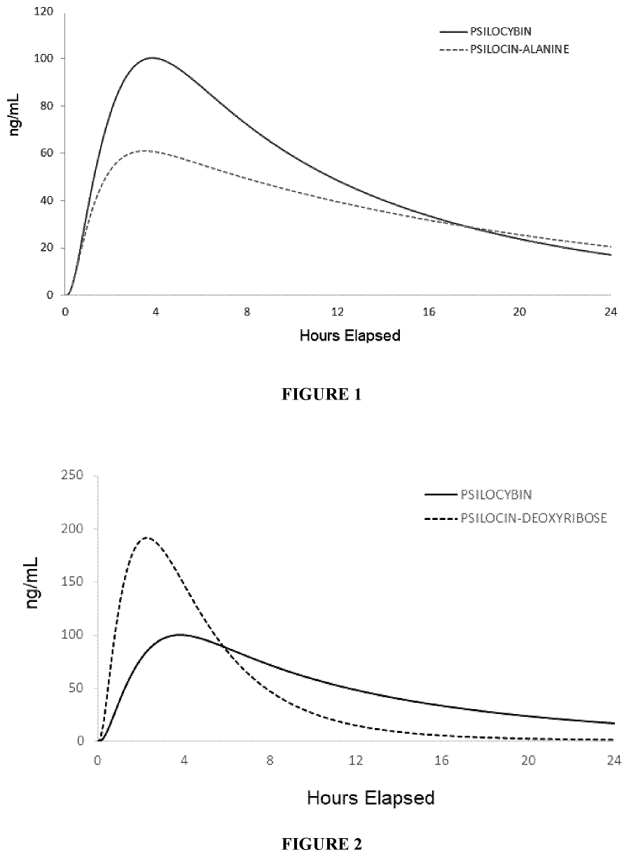

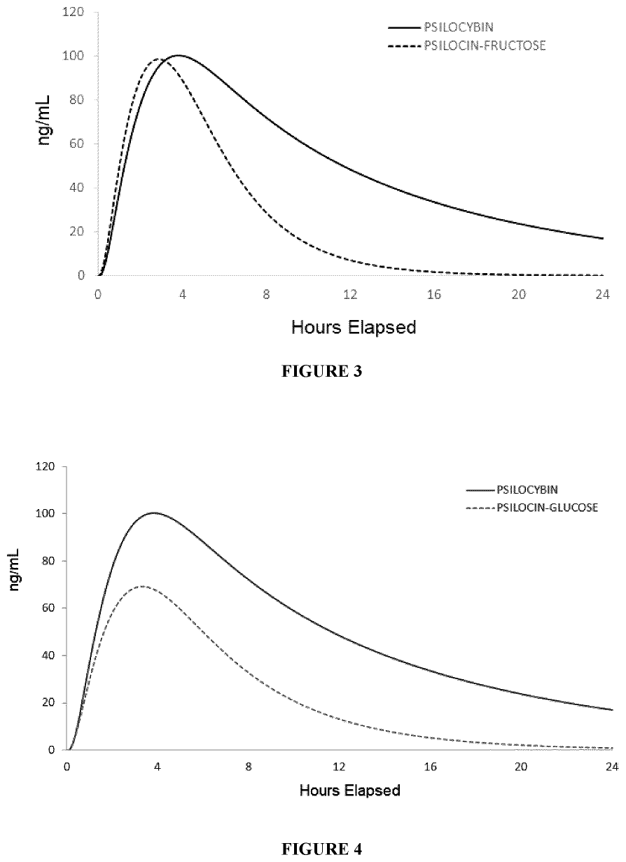

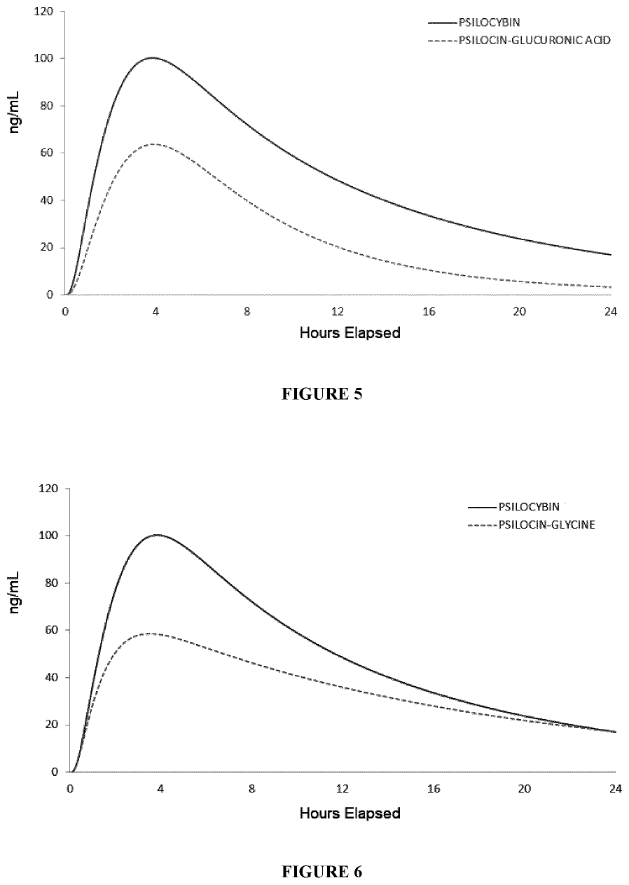

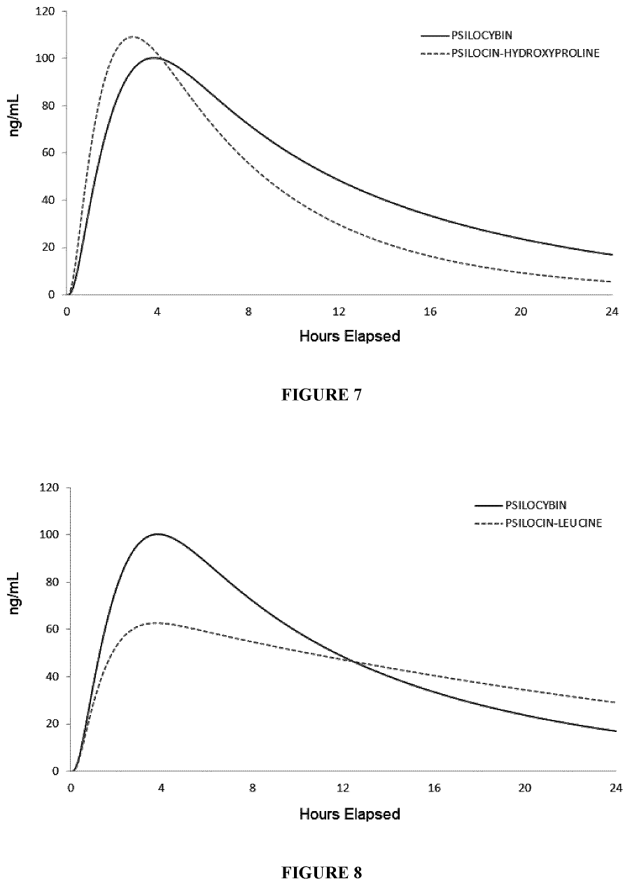

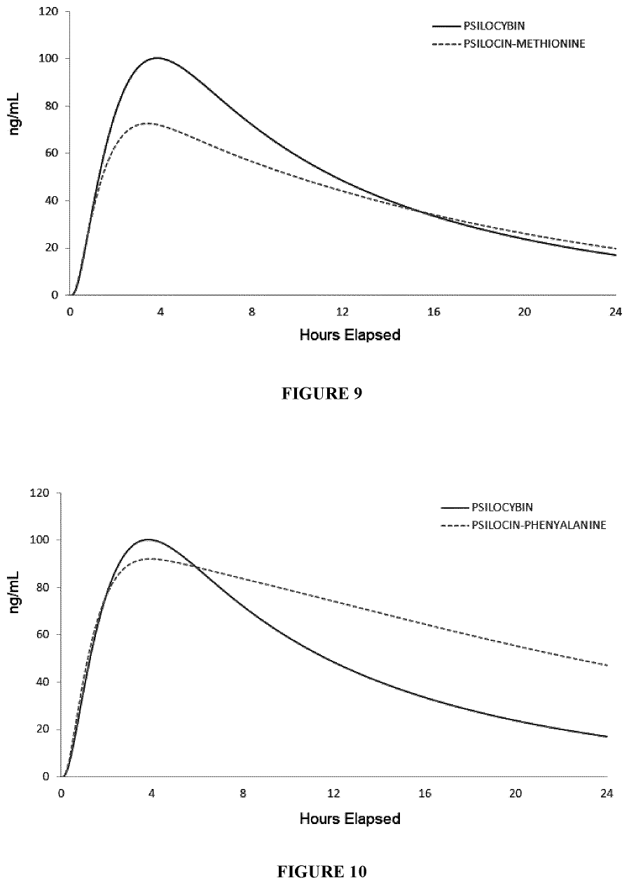

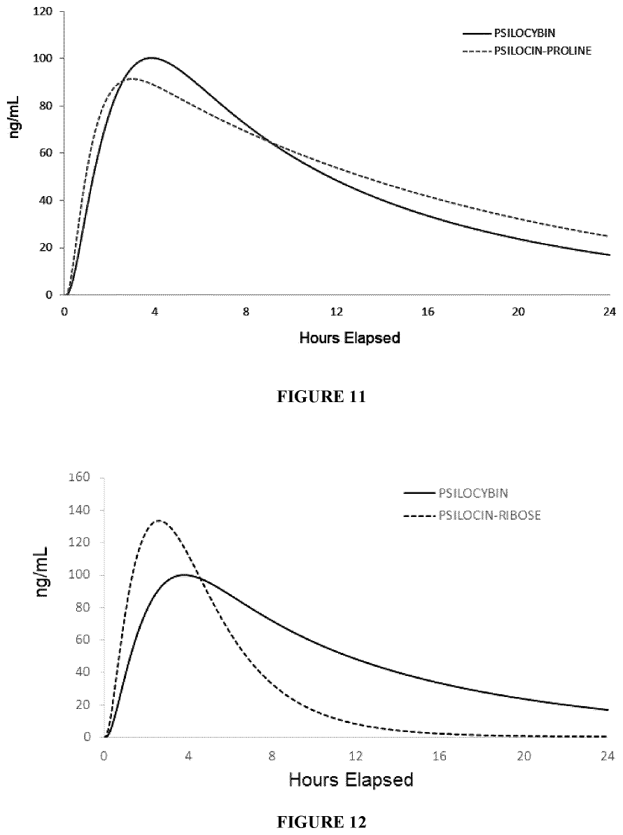

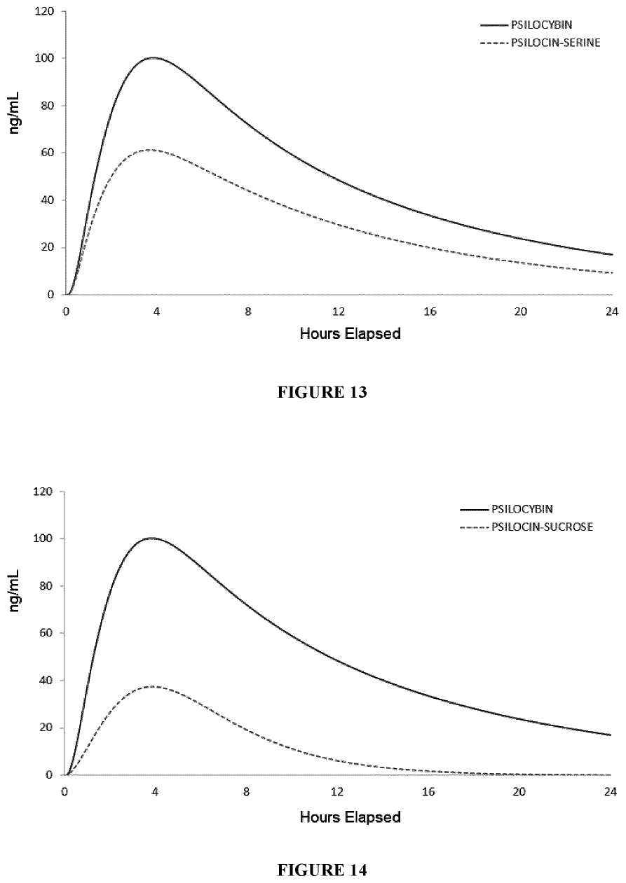

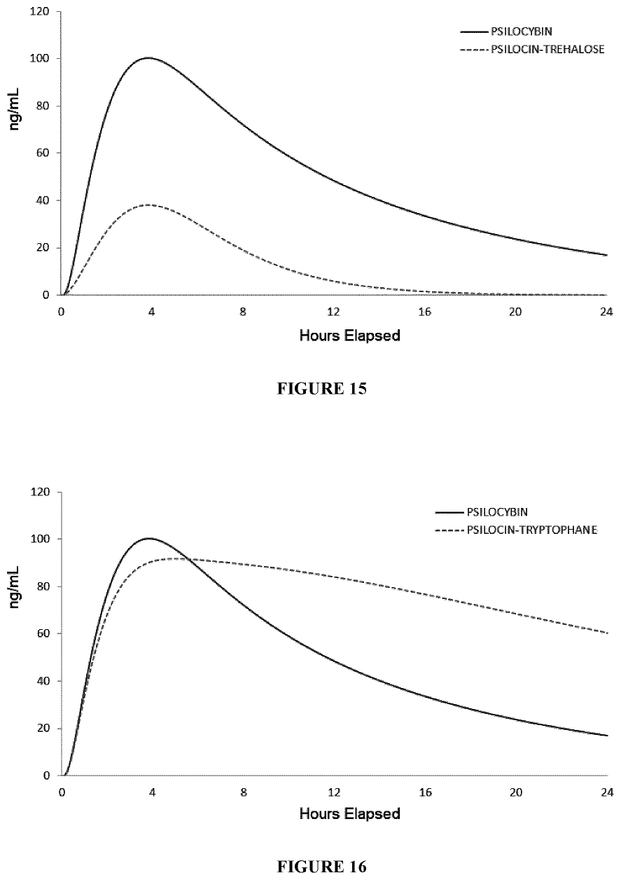

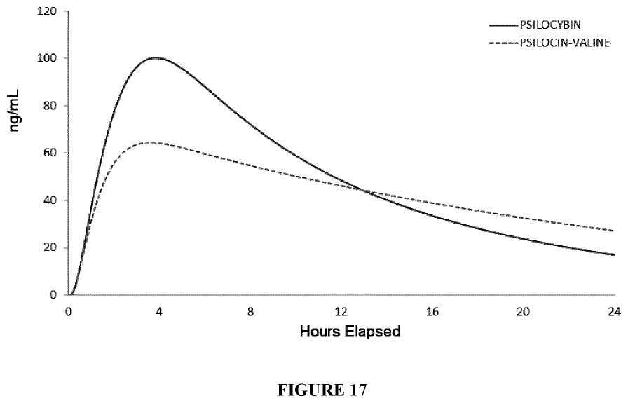

Pharmacokinetic properties of various chemical compounds of Formula I were determined via computer simulation by ADMET Predictor™, a software tool for predicting drug properties. Dosages of 25 mg were provided. The drug plasma levels, as determined by ADMET Predictor™ software simulation, of equal dose of various example chemical entities of Formula I are depicted in FIGS. 1 to 17 of this disclosure. Based on the foregoing parameters, predicted pharmacokinetic properties of various chemical compounds of Formula I were simulated and the results of such simulations are provided in the Tables below (Tables 1 and 2 are intended to be read together and not separately):

TABLE 1

| | | | Fractional | | | |
|---|---|---|---|---|---|---|
| | | | dose | | | |
| | | | absorbed | | | |
| Structure | MW | GP | (%) | Cₘₐₓ | AUC | CL |
| 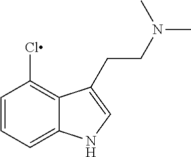 | 204.3 | 1.72 | 94.2 | 51.9 | 438.2 | 35.5 |
| psilocin | | | | | | |
| 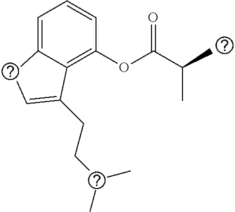 | 275.4 | 1.11 | 87.8 | 60.9 | 918.1 | 15.0 |
| alanine conjugate | | | | | | |
| 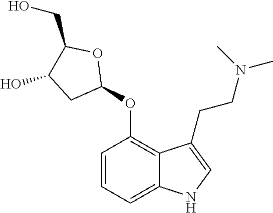 [SMILES: CN(C)CCc1c[nH]c2cccc(O[C@H]3C[C@H](O)[C@@H](CO)O3)c12] | 320.4 | 1.02 | 86.2 | 191.8 | 1109.4 | 16.0 |
| deoxyribose conjugate | | | | | | |
| 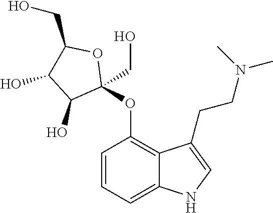 [SMILES: CN(C)CCc1c[nH]c2cccc(O[C@]3(CO)O[C@H](CO)[C@@H](O)[C@@H]3O)c12] | 366.4 | 0.37 | 51.4 | 98.6 | 593.1 | 17.3 |
| fructose conjugate | | | | | | |
| 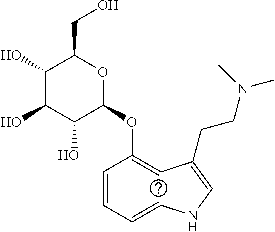 [SMILES: CN(C)CCc1c[nH]ccccc(O[C@@H]2O[C@H](CO)[C@@H](O)[C@H](O)[C@H]2O)c1] | 366.4 | 0.38 | 51.7 | 69.2 | 532.7 | 19.1 |
| glucose conjugate | | | | | | |
| 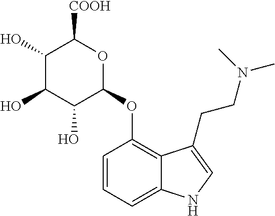 [SMILES: CN(C)CCc1c[nH]c2cccc(O[C@@H]3O[C@H](C(=O)O)[C@@H](O)[C@H](O)[C@H]3O)c12] | 380.4 | 0.26 | 45.8 | 63.6 | 595.8 | 15.5 |
| glucuronic acid | | | | | | |
| conjugate | | | | | | |
| 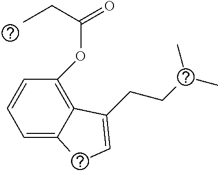 [SMILES: CCC(=O)Oc1cccc2c1C(CCC(C)C)=CC2] | 261.3 | 1.05 | 84.3 | 58.6 | 844.4 | 16.3 |
| glycine conjugate | | | | | | |
| 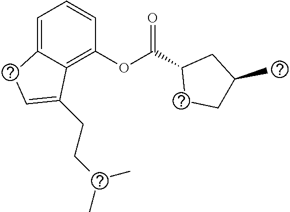 [SMILES: CC(C)CCC1=CCc2cccc(OC(=O)[C@H]3CC[C@H](C)C3)c21] | 317.4 | 0.88 | 83.0 | 109.1 | 965.2 | 17.0 |
| hydroxyproline | | | | | | |
| conjugate | | | | | | |
| 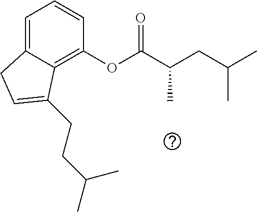 [SMILES: CC(C)CCC1=CCc2cccc(OC(=O)[C@@H](C)CC(C)C)c21] | 317.4 | 1.04 | 92.2 | 62.6 | 1065.1 | 11.7 |
| leucine conjugate | | | | | | |
| 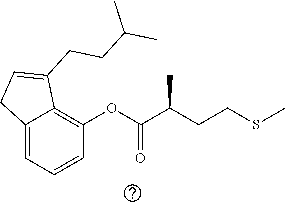 [SMILES: CSCC[C@H](C)C(=O)Oc1cccc2c1C(CCC(C)C)=CC2] | 335.5 | 1.01 | 92.5 | 72.7 | 1032.5 | 14.8 |
| methionine conjugate | | | | | | |
| 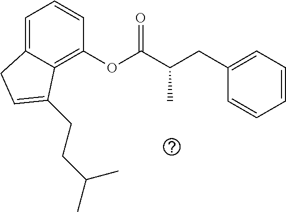 [SMILES: CC(C)CCC1=CCc2cccc(OC(=O)[C@@H](C)Cc3ccccc3)c21] | 351.5 | 1.02 | 95.5 | 92.2 | 1647.7 | 7.8 |
| phenylalanine | | | | | | |
| conjugate | | | | | | |
| 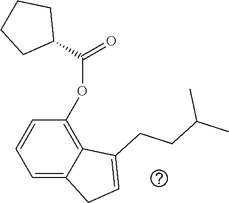 | 301.4 | 1.50 | 96.0 | 91.3 | 1285.6 | 12.6 |
| proline conjugate | | | | | | |
| 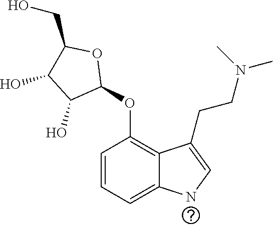 [SMILES: CN(C)CCc1c[nH]c2cccc(O[C@@H]3O[C@H](CO)[C@@H](O)[C@H]3O)c12] | 336.4 | 0.57 | 67.2 | 133.4 | 762.7 | 17.7 |
| ribose conjugate | | | | | | |
| 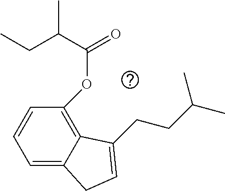 [SMILES: CCC(C)C(=O)Oc1cccc2c1C(CCC(C)C)=CC2] | 291.4 | 0.65 | 68.6 | 61.2 | 735.0 | 17.4 |
| serine conjugate | | | | | | |
| 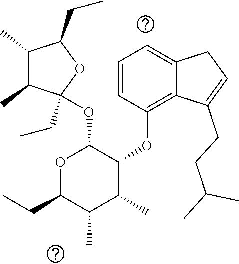 [SMILES: CC[C@H]1O[C@@](CC)(O[C@H]2O[C@H](CC)[C@@H](C)[C@@H](C)[C@H]2Oc2cccc3c2C(CCC(C)C)=CC3)[C@@H](C)[C@@H]1C] | 528.6 | 0.14 | 24.6 | 43.0 | 305.7 | 16.5 |
| sucrose conjugate | | | | | | |
| 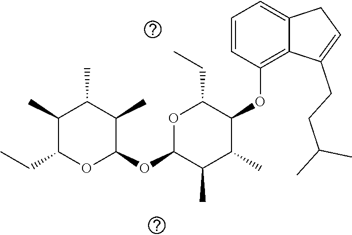 [SMILES: CC[C@H]1O[C@H](O[C@H]2O[C@H](CC)[C@@H](Oc3cccc4c3C(CCC(C)C)=CC4)[C@H](C)[C@H]2C)[C@H](C)[C@@H](C)[C@@H]1C] | 528.6 | 0.12 | 21.9 | 38.1 | 277.2 | 16.2 |
| trehalose conjugate | | | | | | |
| 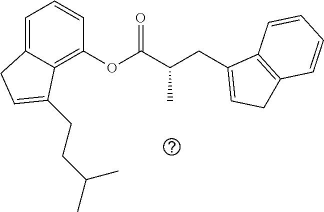 [SMILES: CC(C)CCC1=CCc2cccc(OC(=O)[C@@H](C)CC3=CCc4ccccc43)c21] | 390.5 | 0.73 | 92.9 | 91.7 | 1813.0 | 5.9 |
| tryptophane conjugate | | | | | | |
| 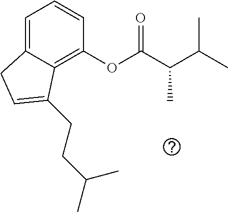 [SMILES: CC(C)CCC1=CCc2cccc(OC(=O)[C@@H](C)C(C)C)c21] | 303.4 | 1.13 | 91.7 | 64.3 | 1053.1 | 12.4 |
| valine conjugate | | | | | | |
| 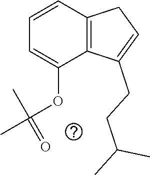 | 284.3 | 0.5 | 68.3 | 100.2 | 1204.4 | 10.8 |
| psilocybin | | | | | | |

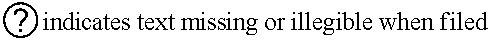

TABLE 2

| | Half-life of | Volume of | Cornea | Skin |
|---|---|---|---|---|
| | conjugate | Distribution | Permeability | Permeability |
| Structure | (hours) | (liters) | (cm/s × 10⁻⁷) | (cm/s x 10⁻⁷) |
| 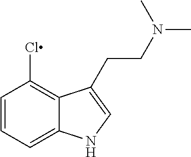 | 4.2 | 216.7 | 157.7 | 1.67 |
| psilocin | | | | |
| 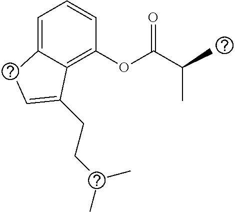 | 11.8 | 253.8 | 129.5 | 2.17 |
| alanine conjugate | | | | |
| 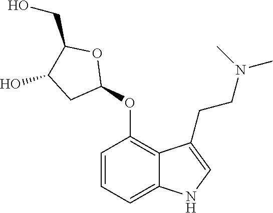 [SMILES: CN(C)CCc1c[nH]c2cccc(O[C@H]3C[C@H](O)[C@@H](CO)O3)c12] | 2.3 | 52.4 | 74.2 | 0.19 |
| deoxyribose conjugate | | | | |
| 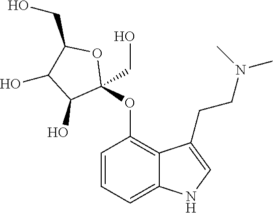 [SMILES: CN(C)CCc1c[nH]c2cccc(O[C@]3(CO)O[C@H](CO)C(O)[C@@H]3O)c12] | 1.9 | 48.0 | 26.7 | 0.09 |
| fructose conjugate | | | | |
| 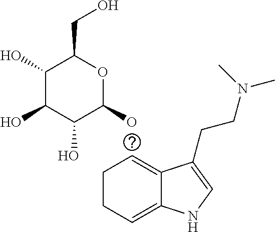 | 3.0 | 81.5 | 33.0 | 0.04 |
| glucose conjugate | | | | |
| 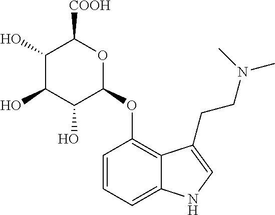 [SMILES: CN(C)CCc1c[nH]c2cccc(O[C@@H]3O[C@H](C(=O)O)[C@@H](O)[C@H](O)[C@H]3O)c12] | 3.9 | 87.4 | 5.7 | 0.01 |
| glucuronic acid conjugate | | | | |
| 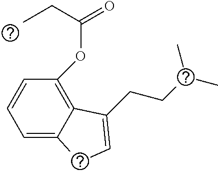 [SMILES: CCC(=O)Oc1cccc2c1C(CCC(C)C)=CC2] | 10.6 | 249.0 | 122.2 | 1.28 |
| glycine conjugate | | | | |
| 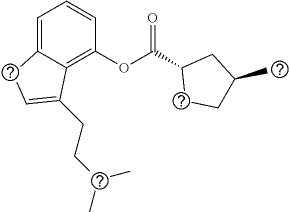 [SMILES: CC(C)CCC1=CCc2cccc(OC(=O)[C@H]3CC[C@H](C)C3)c21] | 4.2 | 102.1 | 124.7 | 0.22 |
| hydroxyproline conjugate | | | | |
| 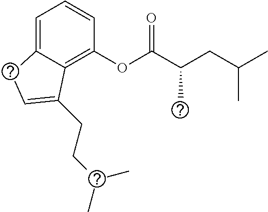 [SMILES: CC(C)CCC1=CCc2cccc(OC(=O)[C@@H](C)CC(C)C)c21] | 15.2 | 258.1 | 160.9 | 9.65 |
| leucine conjugate | | | | |
| 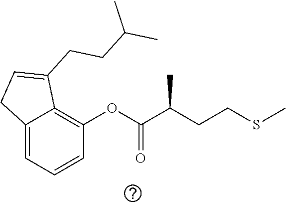 [SMILES: CSCC[C@H](C)C(=O)Oc1cccc2c1C(CCC(C)C)=CC2] | 9.2 | 195.1 | 160.98.7 | 6.95 |
| methionine conjugate | | | | |
| 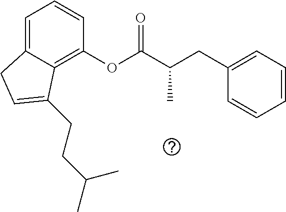 [SMILES: CC(C)CCC1=CCc2cccc(OC(=O)[C@@H](C)Cc3ccccc3)c21] | 16.3 | 182.7 | 254.2 | 5.46 |
| phenylalanine conjugate | | | | |
| 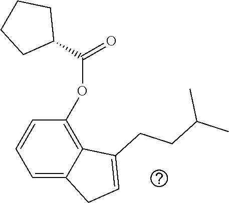 | 10.0 | 182.6 | 235.8 | 1.93 |
| proline conjugate | | | | |
| 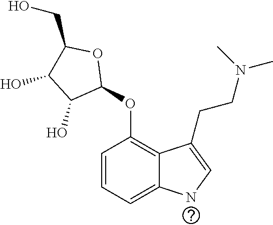 [SMILES: CN(C)CCc1c[nH]c2cccc(O[C@@H]3O[C@H](CO)[C@@H](O)[C@H]3O)c12] | 2.0 | 49.7 | 44.1 | 0.09 |
| ribose conjugate | | | | |
| 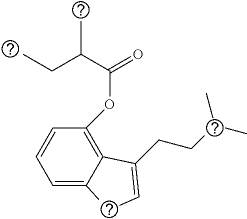 [SMILES: CCC(C)C(=O)Oc1cccc2c1C(CCC(C)C)=CC2] | 6.8 | 170.2 | 65.9 | 0.35 |
| serine conjugate | | | | |
| 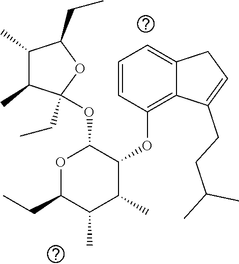 [SMILES: CC[C@H]1O[C@@](CC)(O[C@H]2O[C@H](CC)[C@@H](C)[C@@H](C)[C@H]2Oc2cccc3c2C(CCC(C)C)=CC3)[C@@H](C)[C@@H]1C] | 2.0 | 47.6 | 12.7 | 0.01 |
| sucrose conjugate | | | | |
| 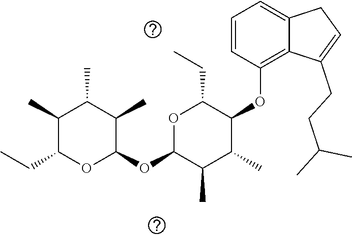 [SMILES: CC[C@H]1O[C@H](O[C@H]2O[C@H](CC)[C@@H](Oc3cccc4c3C(CCC(C)C)=CC4)[C@H](C)[C@H]2C)[C@H](C)[C@@H](C)[C@@H]1C] | 2.0 | 46.6 | 13.7 | 0.01 |
| trehalose conjugate | | | | |
| 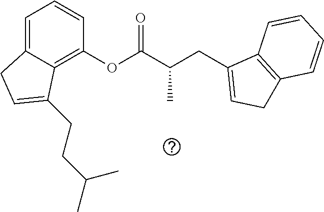 [SMILES: CC(C)CCC1=CCc2cccc(OC(=O)[C@@H](C)CC3=CCc4ccccc43)c21] | 20.4 | 172.2 | 294.2 | 3.71 |
| tryptophan conjugate | | | | |
| 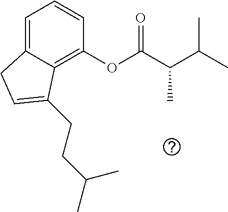 [SMILES: CC(C)CCC1=CCc2cccc(OC(=O)[C@@H](C)C(C)C)c21] | 14.2 | 253.9 | 147.3 | 5.47 |
| valine conjugate | | | | |
| 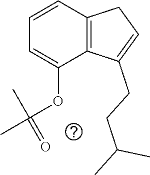 | 6.2 | 96.3 | 9.0 | 0.21 |
| psilocybin | | | | |

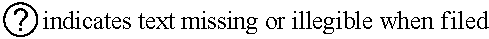

Referring to FIGS. 1 to 17 and Tables 1 and 2 of this disclosure, particular amino acid conjugates and carbohydrate conjugates can be selected according to the desired treatment outcomes.

For example, the following amino acid conjugates all exhibit greater GP and fractional dose absorbed than psilocybin, according to the ADMET Predictor™ model used to generate the data in Tables 1 and 2 of this disclosure: alanine conjugate, deoxyribose conjugate, glycine conjugate, hydroxyproline conjugate, leucine conjugate, methionine conjugate, phenylalanine conjugate, proline conjugate, ribose conjugate, serine conjugate, tryptophan conjugate, and valine conjugate. Without being bound by theory, it is believed that any one of the foregoing amino acid conjugates identified in this paragraph of the disclosure can be used in applications where permeation of chemical compound through the gut is desired.

For example, the following carbohydrate conjugates all exhibit greater Cₘₐₓ than psilocybin, according to the ADMET Predictor™ model used to generate the data in Tables 1 and 2 of this disclosure: deoxyribose conjugate and hydroxyproline conjugate and ribose conjugate. Without being bound by theory, it is believed that any one of the foregoing carbohydrate conjugates identified in this paragraph of the disclosure can be used in applications where high plasma concentration of drug but short duration of action are desired.

For example, the following amino acid conjugates all exhibit greater AUC than psilocybin, according to the ADMET Predictor™ model used to generate the data in Tables 1 and 2 of this disclosure: phenylalanine conjugate, proline conjugate, and tryptophan conjugate. Without being bound by theory, it is believed that any one of the foregoing amino acid conjugates identified in this paragraph of the disclosure can be used in applications where a sustained level of drug plasma concentration over time is desired. The potential use of phenylalanine conjugates and the tryptophan conjugates in applications requiring a sustained level of drug plasma concentration over time is further highlighted by their low clearance in comparison to psilocybin, as predicted using the ADMET Predictor™ model used to generate the data in Tables 1 and 2 of this disclosure. Without being bound by theory, it is believed that such conjugates can be used in dosage regimens where only one dose a day is recommended or required.

For example, the following amino acid conjugates all exhibit greater cornea permeability than psilocybin, according to the ADMET Predictor™ model used to generate the data in Tables 1 and 2 of this disclosure: alanine conjugate, deoxyribose conjugate, fructose conjugate, glucose conjugate, glycine conjugate, hydroxyproline conjugate, leucine conjugate, methionine conjugate, phenylalanine conjugate, proline conjugate, ribose conjugate, serine conjugate, sucrose conjugate, trehalose conjugate, tryptophan conjugate, and valine conjugate. The tryptophan conjugate, proline conjugate, and phenylalanine conjugate in particular are predicted to have high cornea permeability. Without being bound by theory, it is believed that any one of the foregoing amino acid conjugates identified in this paragraph of the disclosure can be used in applications where a chemical compound is delivered to a subject via eye drops.

For example, the following amino acid conjugates all exhibit greater skin permeability than psilocybin, according to the ADMET Predictor™ model used to generate the data in Tables 1 and 2 of this disclosure: alanine conjugate, glycine conjugate, hydroxyproline conjugate, leucine conjugate, methionine conjugate, phenylalanine conjugate, proline conjugate, serine conjugate, tryptophan conjugate, and valine conjugate. The leucine conjugate in particular is predicted to have high skin permeability. Without being bound by theory, it is believed that any one of the foregoing amino acid conjugates identified in this paragraph of the disclosure can be used in applications where the drug is delivered to a subject through the epidermis (e.g. via a patch).

### Synthesis of Examples of Compounds of Formula I

Described below is a non-limiting example of a method of synthesizing (S)-3-(2-(dimethylamino)ethyl)-IH-indol-4-yl 2-amino-3-methylbutanoate hydrochloride.

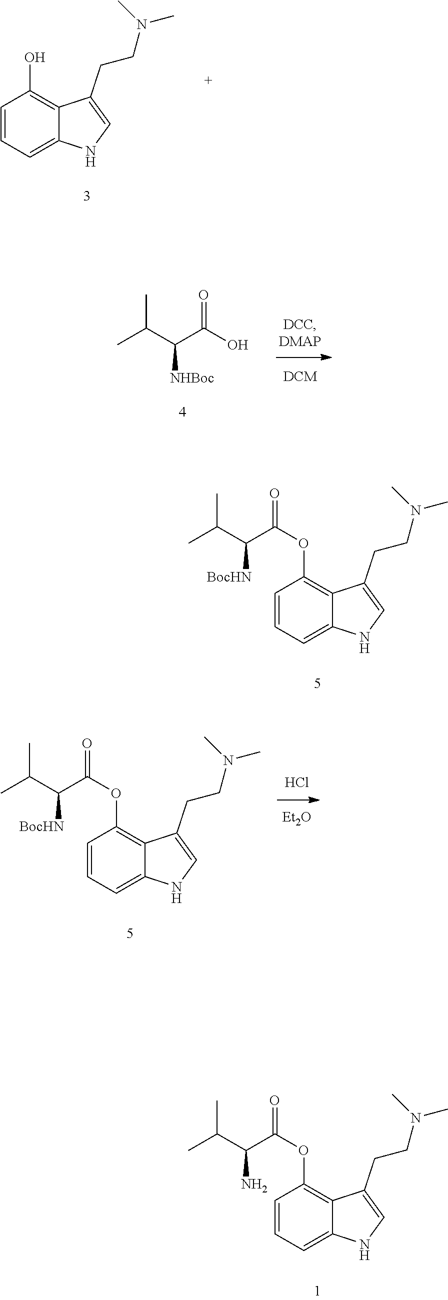

To a cold solution of Boc-L-valine 4 (0.217 g. 1.0 mmol. 2 eq.) in anhydrous dichloromethane (DCM) (10 mL) at 0° C. to 5° C. under nitrogen was added N,N′-Dicyclohexylcarbodiimide (DCC) (0.155 g, 0.75 mmol. 1.5 eq.) followed by 4-Dimethylaminopyridine (DMAP) (0.03 g, 0.25 mmol. 0.5 eq.). The resultant mixture was stirred at 0° C. to 5° C. for 0.5 hour, then a solution of psilocin 3 (0.102 g. 0.5 mmol, 1 eq.) in anhydrous DCM (10 mL) was added. The reaction mixture was stirred at 0° C. to room temperature for 16 hours. The mixture was then filtered to remove solid 1,3-dicyclohexylurea byproduct. The filtrate was concentrated under vacuum. The residue was purified by flash silica gel chromatography eluting with 2% to 4% aqueous NH₄OH (10%)-MeOH in ethyl acetate to yield a clear solid product (0.14 g. yield 70% of compound 5).

The Boc protecting group of compound 5 is then removed in suitable conditions, such as acid. A person of skill in the art would understand that many acids may be used to remove the Boc protecting group, such as HCl. Suitable reaction conditions to remove the Boc protecting group will leave the newly formed ester bond intact.

Described below is a non-limiting example of a method of synthesizing (2S,3R,4S,5S,6R)-2-((3-(2-(dimethylamino)ethyl)-I H-indol-4-yl)oxy)-6-(hydroxymethyl)tetrahydro-2H-pyran-3.4.5-triol.

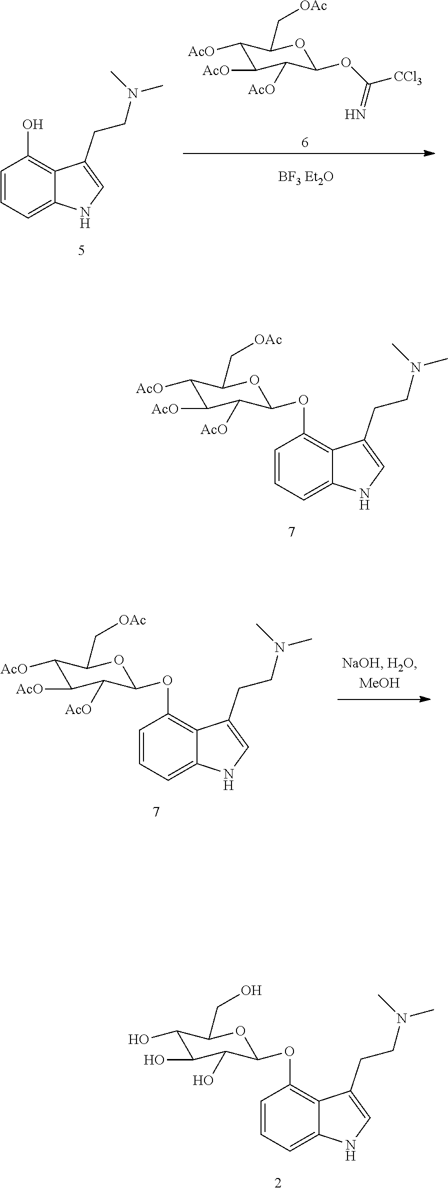

To a solution of Psilocin (3) (0.38 g, 1.86 mmol, 1.0 eq.) and compound 6 (1.07 g, 2.17 mmol. 1.2 eq) in dichloromethane (DCM) (40 mL) was added BF₃OEt₂O (0.7 mL, 5.67 mmol, 3.0 eq) at (5±2°) C slowly. The yellow suspension was stirred at (23±2°) C for 16 h, cooled to (5±2)° C., added sodium bicarbonate (NaHCO₃) aqueous solution (30 mL) slowly and stirred for 30 min. Two layers were separated. The aqueous layer was extracted with DCM (20 mL). The combined organic layer was washed with aqueous NaHCO₃ solution, dried over sodium sulphate (Na₃SO₄), and concentrated to provide a residue that was purified by silica gel chromatography (DCM and DCM:MeOH:NH₄OH=100:3:0.3) to provide compound 7 (0.37 g. yield 37%) as a light pink solid.

To a clear solution of compound 7 (0.11 g, 0.20 mmol, 1 eq) in methanol (MeOH) (250 mL) at (23±2°) C was added aqueous 2N NaOH solution (3.23 mL, 3.23 mmol, 16 eq.) slowly. The resultant mixture was stirred at that temperature for 16 h. The reaction mixture was diluted with water (6 mL) and concentrated under vacuum to remove MeOH. The crude compound was purified by reversed phase C18 column and eluted with water, 50% MeOH in water and 80% MeOH in water to afford (2S,3R,4S,5S,6R)-2-((3-(2-(dimethylamino)ethyl)-I H-indol-4-yl)oxy)-6-(hydroxymethyl)tetrahydro-2H-pyran-3.4.5-triol (2) (0.070 g. yield 95%) as a white solid.

### Method of Use

The compounds described herein are believed to be useful in the treatment of drug resistant depression based on several clinical trials that have been reported using psilocybin itself.

A US STAR*D study has reported that more than half of all patients recruited through primary care and psychiatric clinics fail to achieve remission after first-line antidepressant treatment, and one-third were unable to experience remission after four courses of acute treatment (Rush A J, Trivedi M H, Wisniewski S R, Nierenberg A A, Stewart J W, Warden D, et al. Acute and longer-term outcomes in depressed outpatients requiring one or several treatment steps: a STAR*D report. Am. J. Psychiatry 2006; 163: 1905-17).

In addition to the potential use of these compounds in the treatment of depression, these compounds may also be used to treat tobacco and alcohol addiction. For example, research studies conducted by third party groups on human volunteers have revealed that psilocybin can be used to treat tobacco and alcohol addiction. Moreover, in a controlled clinical environment, psilocybin was safely administered to subjects with OCD, and this drug treatment was found to lead to acute reductions in core OCD symptoms in several subjects (Moreno, F. A., Wiegand, C. B., Taitano, E. K., and Delgado, P. L. “Safety, tolerability, and efficacy of psilocybin in 9 patients with obsessive-compulsive disorder” J. Clin. Psychiatry 2006, 67, 1735-1740). It is believed that the compounds disclosed herein may be used for similar treatments.

Another potential use of these analogs is in the treatment of seizure disorders, including but not limited to infantile seizure disorders such as but not limited to Dravet syndrome (Sourbon, J. et al. “Serotonergic Modulation as Effective Treatment for Dravet Syndrome in a Zebrafish Mutant Model”, ACS Chem. Neurosci. 2016, 7, 588-598).

### Methods of Administration

As contemplated herein, a therapeutically effective amount of a chemical compound described herein is administered to a subject in need thereof. Whether such treatment is indicated depends on the subject case, and is further subject to medical assessment (diagnosis) that takes into consideration signs, symptoms, and/or malfunctions that are present, the risks of developing particular signs, symptoms and/or malfunctions, and other factors.

As contemplated herein, a chemical compound described herein may be administered by any suitable route known in the art. Such routes include oral, buccal, inhalation, topical, mucosal, ophthalmic, sublingual, rectal, vaginal, intracisternal or intrathecal through lumbar puncture, transurethral, nasal, percutaneous, transdermal, and parenteral administration (including intravenous, intramuscular, subcutaneous, intracoronary, intradermal, intramammary, intraperitoneal, intraarticular, intrathecal, retrobulbar, intrapulmonary injection and/or surgical implantation at a particular site). In some embodiments, such routes are selected from the group consisting of mucosal, ophthalmic, transdermal, and intraperitoneal. Parenteral administration may be accomplished using a needle and syringe or using a high pressure technique.

Pharmaceutical compositions include those wherein a chemical compound described herein is present in a sufficient amount to be administered in an effective amount to achieve its intended purpose. The exact formulation, route of administration, and dosage is determined by a qualified medical practitioner in view of the diagnosed condition or disease. Dosage amount and interval can be adjusted individually to provide levels of a chemical compound described herein that is sufficient to maintain the desired therapeutic effects. It is possible that the chemical compound described herein may only require infrequent administration (e.g., monthly or weekly, as opposed to daily) to achieve the desired therapeutic effect.

As contemplated herein, a therapeutically effective amount of a chemical compound described herein adapted for use in therapy varies with the nature of the condition being treated, the length of time that activity is desired, and the age and the condition of the patient, and ultimately is determined by the attendant physician. Dosage amounts and intervals can be adjusted individually to provide plasma levels of the chemical compound that are sufficient to maintain the desired therapeutic effects including the use of micro-dosing. The desired dose conveniently may be administered in a single dose, or as multiple doses administered at appropriate intervals, for example as one, two, three, four, or more subdoses per day. Multiple doses often may be desired or required.

As contemplated herein, the chemical compounds described herein may be administered in admixture with a pharmaceutical carrier selected with regard to the intended route of administration and standard pharmaceutical practice. Pharmaceutical compositions for use in accordance with the chemical compounds described herein are formulated in a conventional manner using one or more physiologically acceptable carriers comprising excipients and auxiliaries that facilitate processing of the compounds described herein.

Water is a preferred carrier when the chemical compounds described herein are administered intravenously. Saline solutions and aqueous dextrose and glycerol solutions may also be used as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, ethoxy diglycol, isopropyl myristate, oleic acid, triglycerides, polysorbate, caprylocaproyl polyoxyl-8 glycerides, water, ethanol, and the like. The present compositions, if desired, can also contain minor amounts of wetting or emulsifying agents, or pH buffering agents.

These pharmaceutical compositions may be manufactured, for example, by conventional mixing, dissolving, granulating, dragee-making, emulsifying, encapsulating, entrapping, cocrystallization, milling, coacervation, precipitation or lyophilizing processes. Proper formulation is dependent upon the route of administration chosen. When a therapeutically effective amount of a chemical compound described herein is administered orally, the composition typically is in the form of a tablet, capsule, powder, solution, nanoparticle, nanocrystal, liposome, self-emulsifying drug delivery system, self-micro emulsifying drug delivery system, micro emulsion emulsion, suspension, or elixir. If administered in tablet or capsule form, the composition additionally can contain a solid carrier and shell made from gelatin or cellulose derivate such as hydroxypropyl methyl cellulose. The tablet, capsule, and powder contain about 0.01% to about 95%, and preferably from about 1% to about 50%, of a chemical compound described herein. If administered in liquid form, a liquid carrier, such as water, petroleum, or oils of animal or plant origin, can be added. The liquid form of the composition can further contain physiological saline solution, dextrose or other saccharide solutions, cosolvents, surfactants, antioxidants or glycols. If administered in liquid form, the composition contains about 0.1% to about 90%, and preferably about 1% to about 50%, by weight, of a chemical compound described herein.

If a therapeutically effective amount of a chemical compound described herein is administered by intravenous, cutaneous, or subcutaneous injection, the composition is in the form of a sterile, pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle, pH adjusting buffer, and antioxidants.

The chemical compounds described herein may be readily combined with pharmaceutically acceptable carriers well-known in the art. Such carriers enable the active agents to be formulated as tablets (such as orally disintegrating tablets or orally dissolving films), pills, dragees, capsules, liquids, gels, syrups, slurries, suspensions and the like, for oral ingestion by a patient to be treated.

Pharmaceutical preparations for oral use can be obtained by adding a chemical compound described herein to a solid excipient, with or without grinding the resulting mixture, and processing the mixture of granules, after adding suitable auxiliaries, if desired, to obtain capsules, tablets or dragee cores. Suitable excipients include, for example, fillers and cellulose preparations. If desired, disintegrating agents can be added.

A chemical compound described herein may be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending agents, stabilizing agents, dispersing agents, pH adjusting buffers, and any combination thereof.

Pharmaceutical compositions for parenteral administration include aqueous solutions of the active agent in water-soluble form. Additionally, suspensions of a chemical compound described herein can be prepared as appropriate oily injection suspensions. Suitable lipophilic solvents or vehicles include fatty oils or synthetic fatty acid esters. Aqueous injection suspensions can contain substances which increase the viscosity of the suspension.

In some embodiments, the suspension also can contain suitable stabilizers or agents that increase the solubility of the compounds and allow for the preparation of highly concentrated solutions. Alternatively, a present composition can be in powder form for constitution with a suitable vehicle (e.g., sterile pyrogen-free water) before use.

A chemical compound described herein also may be formulated in rectal compositions, such as suppositories or retention enemas (e.g., suppositories or retention enemas containing conventional suppository bases). In addition to the formulations described previously, a chemical compound described herein also can be formulated as a depot preparation. Such long-acting formulations can be administered by implantation (for example, subcutaneously or intramuscularly) or by intramuscular injection. Thus, for example, a chemical compound described herein may be formulated with suitable polymeric or hydrophobic materials (for example, as an emulsion in an acceptable oil) or ion exchange resins.

A chemical compound described herein may be administered orally, buccally, or sublingually in the form of tablets (such as orally disintegrating tablets or orally dissolving films) containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The chemical compounds described herein also may be injected parenterally, for example, intravenously, intramuscularly, subcutaneously, or intracoronarily. For parenteral administration, the chemical compounds described herein may be best used in the form of a sterile aqueous solution which can contain other substances, for example, salts or monosaccharides, such as mannitol or glucose, to make the solution isotonic with blood. The chemical compounds described herein also may be inhaled as solution, suspension, or powder. The chemical compounds described herein also may be administered intra nasally as solution, suspensions, or powder.

### GENERAL

It is contemplated that any part of any aspect or embodiment discussed in this specification may be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

Unless defined otherwise, all technical and scientific terms used herein have the same meaning as is commonly understood by one of ordinary skill in the art to which this invention belongs. In addition, any citation of references herein is not to be construed nor considered as an admission that such references are prior art to the present invention.

The scope of the claims should not be limited by the example embodiments set forth herein, but should be given the broadest interpretation consistent with the description as a whole.

What is claimed is:

1. A chemical compound of Formula 1 selected from the group consisting of an amino acid ester and a carbohydrate conjugate:

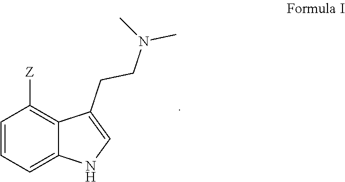

2. The chemical compound as claimed in claim 1, wherein Z is selected from the group consisting of C₆H₁₁O₆—, C₁₂H₂₁O₁₁—, C₅H₉O₅—, and C₅H₉O₄—.

3. The chemical compound as claimed in claim 2, wherein Z is C₆H₁₁O₆— and is selected from the group consisting of glucose, allose, altrose, mannose, gulose, idose, galactose, talose, psicose, fructose, sorbose, and tagatose, and derivatives of any one thereof.

4. The chemical compound as claimed in claim 2, wherein the chemical compound is a glucose conjugate.

5. The chemical compound as claimed in claim 2, wherein Z is C₁₂H₂₁O₁₁— and is selected from the group consisting of trehalose, and sucrose, and derivatives of any one thereof.

6. The chemical compound as claimed in claim 2, wherein Z is C₅H₉O₅— and is selected from the group consisting of ribose, arabinose, xylose, lyxose, ribulose, and xylulose, and derivatives of any one thereof.

7. The chemical compound as claimed in claim 2, wherein Z is C₅H₉O₄— and is selected from the group consisting of ribose, arabinose, xylose, lyxose, ribulose, and xylulose, and derivatives of any one thereof.

8. The chemical compound as claimed in claim 1, wherein: (i) Z is selected from the group consisting of C₆H₁₃N₄O₂—, C₆H₉N₃O₂—, C₆H₁₃N₂O₂—, C₄H₈NO₄—, C₅H₃NO₄—, C₃H₇NO₃—, C₄H₈NO₃—, C₄H₇N₂O₃—, C₅H₉N₂O₃—, C₃H₆NO₂X—, C₂H₄NO₂—, C₅H₈NO₂—, C₅H₈NO₃—, C₃H₆NO₂—, C₅H₁₀NO₂—, C₆H₁₂NO₂—, C₅H₁₀NO₂S—, C₉H₁₀NO₂—, C₉H₁₀NO₃—, and C₁₁H₁₁N₂O₂—, and derivatives of any one thereof; and (ii) X can be selected from the group consisting of S and Se.

9. The chemical compound as claimed in claim 8, wherein the chemical compound is selected from the group consisting of a proline ester, a valine ester, and derivatives of any one thereof.

10. The chemical compound as claimed in claim 8, wherein the chemical compound is a valine ester.

11. The chemical compound as claimed in claim 1, wherein the chemical compound is selected from the group consisting of an alanine conjugate, deoxyribose conjugate, glycine conjugate, hydroxyproline conjugate, leucine conjugate, methionine conjugate, phenylalanine conjugate, proline conjugate, ribose conjugate, serine conjugate, tryptophan conjugate, and valine conjugate.

12. The chemical compound as claimed in claim 1, wherein the chemical compound is selected from the group consisting of a deoxyribose conjugate and hydroxyproline conjugate and ribose conjugate.

13. The chemical compound as claimed in claim 1, wherein the chemical compound is selected from the group consisting of a phenylalanine conjugate, proline conjugate, and tryptophan conjugate.
